(19) United States
(12) Patent Application Publication
(10) Pub. No.: US 2024/0114900 A1
(43) Pub. Date: Apr. 11, 2024

(54) ECO-FRIENDLY CAPSULE MANUFACTURING METHOD FOR STABLE LOADING OF ANTISEPTIC DISINFECTANT AND ECO-FRIENDLY CAPSULE MANUFACTURED THEREFROM

(71) Applicants: KYUNGPOOK NATIONAL UNIVERSITY INDUSTRY-ACADEMIC COOPERATION FOUNDATION, Daegu (KR); SEOUL NATIONAL UNIVERSITY R&DB FOUNDATION, Seoul (KR)

(72) Inventors: Moon Kyu KWAK, Daegu (KR); Bong Su KANG, Daegu (KR); Hosup JUNG, Incheon (KR)

(21) Appl. No.: 18/276,596

(22) PCT Filed: Feb. 9, 2022
(86) PCT No.: PCT/KR2022/001950

§ 371 Date: Aug. 9, 2023

(30) Foreign Application Priority Data
Feb. 9, 2021 (KR) 10-2021-0018573
Jul. 7, 2021 (KR) 10-2021-0088968

(51) Int. Cl. A01N 25/28; A01N 25/10; A01P 1/00; B01J 13/18; B01J 13/20

(52) U.S. Cl. CPC A01N 25/28; A01N 25/10; A01P 1/00; B01J 13/18; B01J 13/206

(57) ABSTRACT

A method for manufacturing an eco-friendly capsule includes: disposing a water phase material containing chitosan at a first site, and disposing an oil phase material at a second site and a third site, on a microfluidic chip; moving the water phase material and the oil phase material to a reaction site according to a predetermined flow ratio to produce an emulsion containing droplets having a predetermined diameter; mixing the emulsion with a water phase solution in a mixing tank; and producing chitosan beads by the reaction of the emulsion with the water phase solution.

### TECHNICAL FIELD

The present invention relates to a method for manufacturing an eco-friendly capsule for stable loading of a freezing-resistant disinfectant and an eco-friendly capsule manufactured therefrom, and more specifically, to a method for manufacturing an eco-friendly capsule for stable loading of a freezing-resistant disinfectant, which may maintain the effect of the freezing-resistant disinfectant for a long period of time, and an eco-friendly capsule manufactured therefrom.

### BACKGROUND ART

The government and livestock authorities have continuously promoted quarantine measures to prevent the recurrence of livestock infectious diseases that occur repeatedly, but it has been pointed out that the fundamental quarantine measures are insufficient, as shown by the fact that the amount of damage caused by livestock infectious diseases from 2000 to 2017 reached about KRW 44 trillion, and AI/foot-and-mouth disease also recurred in 2016 and 2017. In addition, deadly viruses such as African swine fever are transmitted through wild boars, causing serious damage to pig farms.

The scale of damage caused by livestock infectious diseases is so enormous that it accounts for 20% of the total production of livestock farms, and as breeding is grouped, the scale of damage caused by diseases is increasing, and in particular, due to market opening, the damage caused by the inflow of malignant infectious diseases such as AI/foot-and-mouth disease and African swine fever from abroad is increasing, so that there is an urgent need for measures to prevent and prevent the spread of, livestock diseases.

More than tens of billions of won in quarantine costs are invested every year to prevent these livestock infectious diseases, and the national compensation for livestock infectious diseases continues to increase; and in the prevention of livestock infectious diseases, biosecurity is the first line of farm quarantine, and vaccination is the final line of defense, but in the case of infectious livestock diseases for which vaccination is not possible, effective biosecurity requires the use of effective disinfectants as well as mechanical transmission blocking (control of access to the farm by vehicles and outsiders).

Therefore, for application to the domestic situation, as the need to develop a freezing-resistant disinfectant that may be used without changing its chemical and physical properties even in the winter season (freeze-up) has emerged, various freezing-resistant disinfectants have been developed, but there is a problem of using environmental pollutants such as antifreeze for a non-freezing effect in the winter season and a problem of poor durability of efficacy.

### PRIOR ART DOCUMENT

### Patent Document

Korean Patent No. 10-2106811

### DISCLOSURE

### Technical Problem

The present invention has been made to solve the above problems, and it is an object of the present invention to provide a method for manufacturing an eco-friendly capsule for efficient loading of a freeze-resistant disinfectant, which may maintain the efficacy of the freezing-resistant disinfectant for a long period of time by manufacturing a capsule loaded with the freezing-resistant disinfectant and allowing the loaded freezing-resistant disinfectant component to be slowly released.

In addition, it is another object of the present invention to provide an eco-friendly disinfectant capsule manufactured according to a method for manufacturing an eco-friendly capsule, which may stably load a freeze-resistant disinfectant without environmental pollution due to disinfectant spraying and toxicity by manufacturing a capsule loaded with the freeze-resistant disinfectant using eco-friendly materials.

### Technical Solution

In order to achieve the above objects, the method for manufacturing an eco-friendly capsule according to an embodiment of the present invention includes the steps of: disposing a water phase material containing chitosan at a first site, and disposing an oil phase material at a second site and a third site, on a microfluidic chip; moving the water phase material and the oil phase material to a reaction site according to a predetermined flow ratio to produce an emulsion containing droplets having a predetermined diameter; mixing the emulsion with a water phase solution in a mixing tank; and producing chitosan beads by the reaction of the emulsion with the water phase solution.

In this case, the oil phase material may be a material composed by mixing oil and a first surfactant according to a predetermined ratio, and the water phase solution may be a material composed by mixing a cross-linking agent and a second surfactant according to a predetermined ratio.

In addition, the step of producing chitosan beads may comprise the steps of: generating oil emulsification at the interface between the emulsion and the water phase solution; phase-inverting the first surfactant remaining on the surface of the droplets into the second surfactant; and producing beads by the reaction of the droplets with the cross-linking agent.

In addition, in the step of producing the beads, the reaction of the droplets with the cross-linking agent may be promoted by the phase-inverting step.

In addition, in the step of producing the beads, the reaction of the droplets with the cross-linking agent may be promoted by centrifugation.

In addition, it may comprise the steps of: moving oil remaining in the water phase solution to the interface by the second surfactant; and curing and precipitating the produced beads according to the continuous progress of cross-linking.

In addition, it may comprise the steps of: separating and washing the precipitated beads from the mixing tank; and drying the washed beads at room temperature.

In addition, the predetermined flow ratio between the water phase material and the oil phase material may be in the range of 1 to 5-20, and the diameter of the droplets may be within 50 m.

### Advantageous Effects

According to one aspect of the present invention described above, by loading a freezing-resistant disinfectant and allowing the freezing-resistant disinfectant component to be slowly released, the effect of the loaded freezing-resistant disinfectant may be maintained for a long period of time.

In addition, an eco-friendly capsule for stable loading of a freezing-resistant disinfectant without environmental pollution and toxicity may be provided by manufacturing using eco-friendly materials, and an eco-friendly capsule may be provided by establishing optimal particle manufacturing conditions that have a virucidal effect and minimize the toxicity of the cross-linking agent.

### DESCRIPTION OF DRAWINGS

FIG. 1 is a flow chart illustrating the method for manufacturing an eco-friendly capsule according to the present invention,

FIG. 2 is a view illustrating the state in which the emulsion is produced by the method for manufacturing an eco-friendly capsule according to the present invention,

FIGS. 3A to 4 are views illustrating the state in which beads are produced by the method for manufacturing an eco-friendly capsule according to the present invention,

FIGS. 5A to 5C are views illustrating the state in which beads produced by the method for manufacturing an eco-friendly capsule according to the present invention are washed and dried, and

FIGS. 6A to 6D are views illustrating the efficacy of beads produced by varying the content of the cross-linking agent in the method for manufacturing an eco-friendly capsule according to the present invention.

### BEST MODE

The detailed description of the present invention described below refers to the appended drawings, which shows, by way of illustration, specific embodiments in which the present invention may be practiced. These embodiments are described in sufficient detail to enable one skilled in the art to practice the present invention. It should be understood that the various embodiments of the present invention are different from each other but are not necessarily mutually exclusive. For example, specific shapes, structures, and characteristics described herein may be embodied in another embodiment without departing from the spirit and scope of the present invention in connection with one embodiment. In addition, it should be understood that the location or arrangement of individual components within each disclosed embodiment may be changed without departing from the spirit and scope of the present invention. Thus, the detailed description described below is not intended to be taken in a limiting sense, and the scope of the present invention, if properly described, is limited only by the appended claims, along with all equivalents to the scope claimed in the claims. Similar reference numerals in the drawings refer to the same or similar function throughout the various aspects.

Hereinafter, preferred embodiments of the present invention will be described in more detail with reference to the drawings.

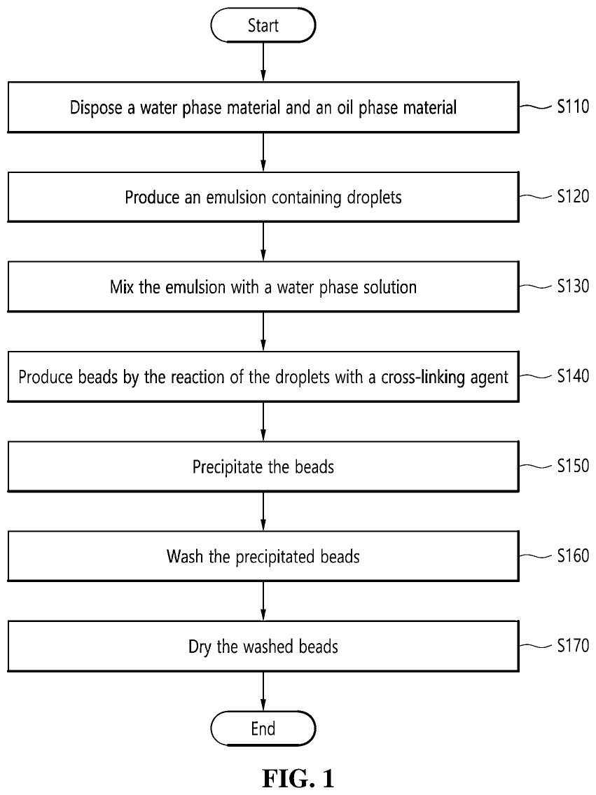

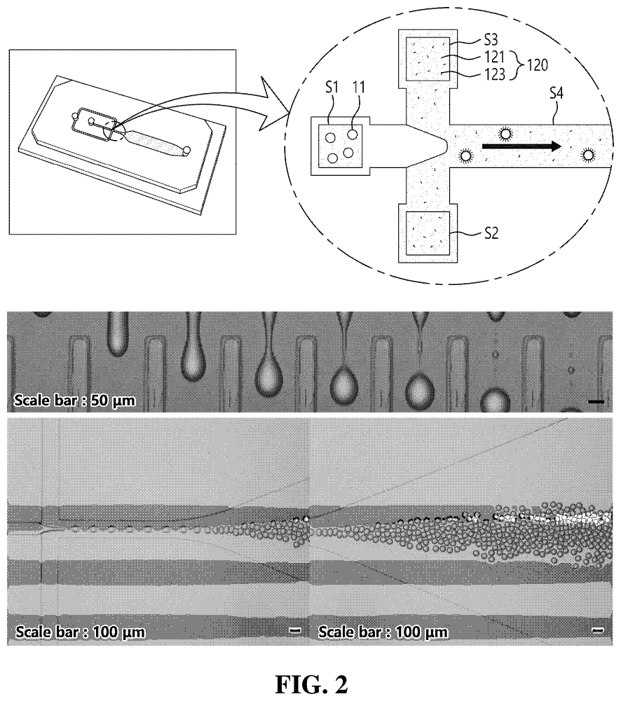

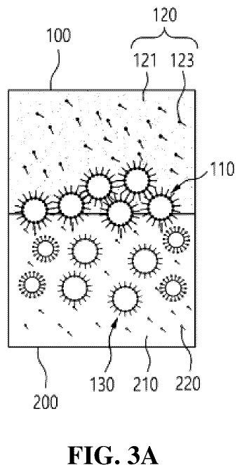

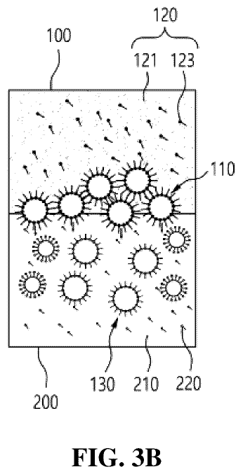

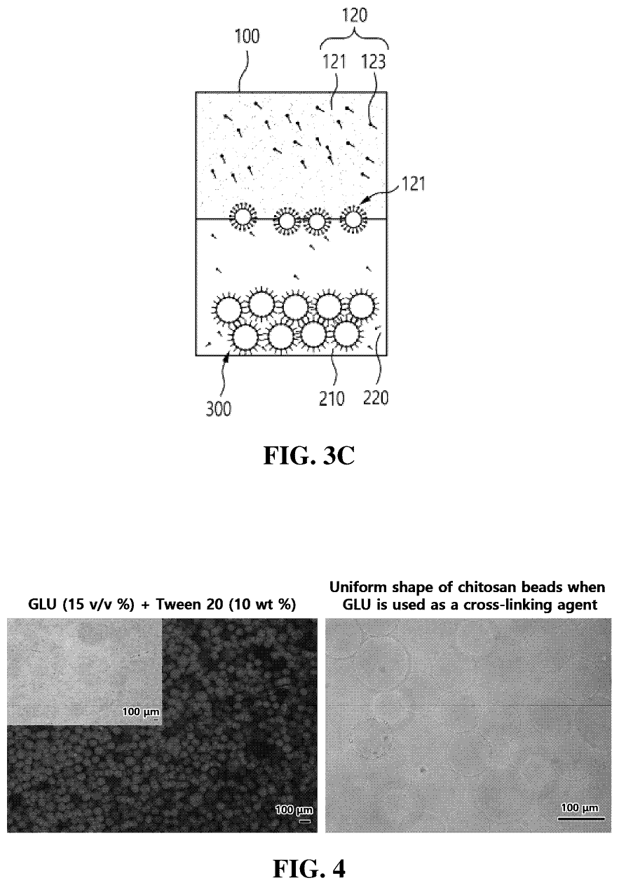

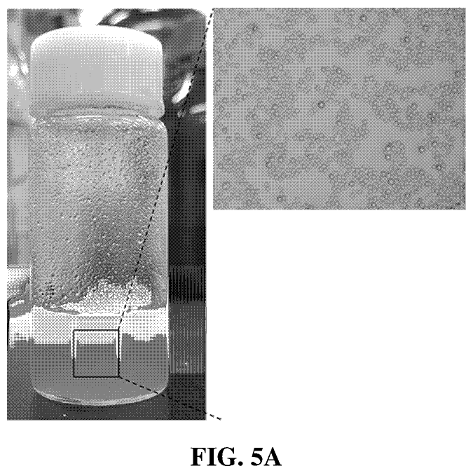

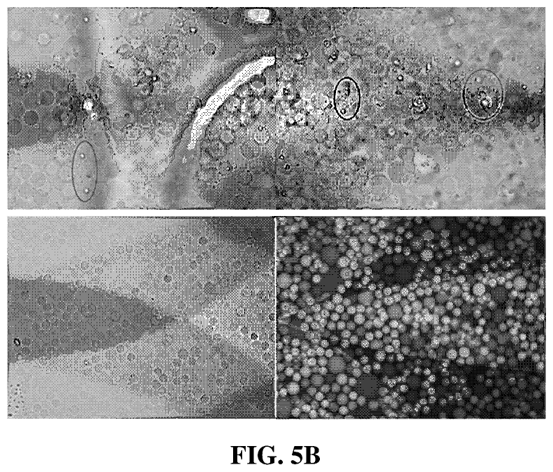

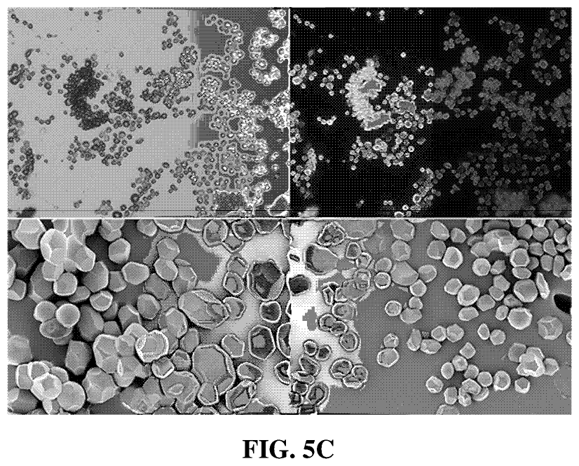

FIG. 1 is a flow chart illustrating the method for manufacturing an eco-friendly capsule according to the present invention, FIG. 2 is a view illustrating the state in which the emulsion is produced by the method for manufacturing an eco-friendly capsule according to the present invention, FIGS. 3A to 4 are views illustrating the state in which beads are produced by the method for manufacturing an eco-friendly capsule according to the present invention, and FIGS. 5A to 5C are views illustrating the state in which beads produced by the method for manufacturing an eco-friendly capsule according to the present invention are washed and dried.

First, on the microfluidic chip, the water phase material 11 containing chitosan is disposed at the first site S1, and the oil phase material 120 is disposed at the second site S2 and the third site S3 (S110).

In this embodiment, as shown in FIG. 2, it has been described that the oil phase material 120 is disposed at the second site S2 and the third site S3, respectively, but it may be disposed at one site.

The water phase material 11 in the present invention is a composition composed of purified water (DI water), freezing-resistant disinfectant, acetic acid and chitosan, wherein the purified water and the freezing-resistant disinfectant may be mixed at a volume ratio of 1:1, the acetic acid may be composed of 1% v/v, and the chitosan may be composed of 1 to 3 wt %.

On the one hand, the oil phase material 120 is a material composed of mineral oil 121 and first surfactant 123, wherein the first surfactant 123 is composed of 5 to 20 wt %, and Span 80 is used as the first surfactant 123 in this embodiment.

Subsequently, the emulsion 100 containing droplets 110 having a predetermined diameter is produced by moving the water phase material 11 and the oil phase material 120 to the reaction site S4 according to a predetermined flow ratio (S120).

In this case, the predetermined flow ratio between the water phase material 11 and the oil phase material 120 is in the range of 1 to 5-20, and the diameter of the droplets 110 contained in the emulsion 100 may be size-adjusted within 50 m according to the flow ratio. In addition, as shown in FIG. 2, the droplets 110 produced while the water phase material 11 passes through the first site S1 of the microfluidic chip are mixed with the water phase material 120 at the reaction site S4, thereby producing an emulsion 100. The emulsion 100 thus produced is moved in the direction of the arrow in FIG. 2, and the emulsion 100 thus moved may be accommodated in a mixing tank (not shown). The mixing tank (not shown) may be connected to one end of the reaction site S4 of the microfluidic chip through a separate connection means.

Then, the emulsion 100 is mixed with the water phase solution 200 in the mixing tank (S130).

In this case, the water phase solution 200 is a material composed by mixing the cross-linking agent 210 and the second surfactant 220 according to a predetermined ratio, wherein glutaraldehyde (GLU) is used as the cross-linking agent 210, but its pH may be adjusted to 2.0 and composed of 1 to 4% v/v, and Tween 20 is used as the second surfactant 220, but may be composed of 5 to 10 wt %.

Next, chitosan beads 130 are produced by the reaction between the emulsion 100 and the water phase solution 200 mixed (S140).

The step of producing chitosan beads (S140) may comprise the steps of: generating oil emulsification at the interface between the emulsion 100 and the water phase solution; phase-inverting the first surfactant 123 remaining on the surface of the droplets 110 into the second surfactant 220; and producing beads 130 by the reaction of the droplets 130 with the cross-linking agent 210.

In the step of phase-inverting the first surfactant 123 remaining on the surface of the droplets 110 into the second surfactant 220, as described above, Tween 20 was used as the second surfactant 220 in this embodiment, which is based on the characteristics of the type of surfactant used for the phase inversion, and it is preferable to use Tween 20 as the second surfactant 220 in this embodiment because the separation and precipitation reaction of the chitosan beads 130 proceeds faster than when sodium dodecyl sulfate (SDS) instead of Tween 20 is used.

As shown in in FIG. 3A, the droplets 110 in the emulsion 100 is droplets 110 combined with the first surfactant 123, and as shown in FIG. 3B, when the emulsion 100 and the water phase solution 200 are mixed, the first surfactant 123 remaining on the surface of the droplets 110 is converted into the second surfactant 220 according to the above-described phase inversion, and from this, cross-linking between the droplets 110 and the cross-linking agent 210 may be promoted.

In FIGS. 3A to 3C, for convenience of description, the produced chitosan beads 130 and the cured and precipitated beads 300 according to the continuous progress of cross-linking are described with different reference numerals, but the produced chitosan beads 130 and the precipitated beads 300 may be the same bead with a difference only in the degree of curing.

In addition, as shown in FIG. 4, the chitosan beads 130 according to this embodiment may be produced in a uniform size by using GLU as the cross-linking agent 210.

Specifically, the maintenance of the shape of the chitosan beads 130 may vary depending on the type of cross-linking agent 210, and it was confirmed that when using tripolyphosphate (TPP) among the types of cross-linking agent 210, the shape of the chitosan beads 130 was not maintained. As shown in FIG. 4, it was confirmed that the chitosan beads 130 according to this embodiment may be produced in a uniform size by using GLU as the cross-linking agent 210, and thus, it is preferable to use GLU as the cross-linking agent 210.

In this case, the reaction between the droplets 110 and the cross-linking agent 210 may be promoted by the above-described phase-inverting step.

In the steps of generating oil emulsification at the interface between the emulsion 100 and the water phase solution, the oil 121 remaining in the water phase solution 200 moves to the interface by reaction with the second surfactant 220, as shown in FIGS. 3B and 3C while the chitosan beads 130 are produced.

Thereafter, the produced beads 130 are cured and precipitated according to the continuous progress of cross-linking (S150). As shown in FIG. 3B, the beads 130 are produced, and the produced beads are hard cured and precipitated as the cross-linking with the cross-linking agent 210 continues to proceed. In this case, centrifugation may be used to promote the reaction between the droplets 110 and the cross-linking agent 210, and when the centrifugation is performed at 1200 rpm for 30 minutes to proceed with cross-linking, the formation rate of the chitosan beads 130 is improved and the precipitation proceeds faster than in the stationary state, so that separation may be easily performed.

Then, the precipitated beads 300 are separated from the mixing tank and washed (S160). FIG. 5A shows the appearance of the precipitated beads 300 according to the present invention, and the particle size of the precipitated beads 300 separated from the mixing tank may be formed in 24.65±3.94 μm.

In addition, the top image of FIG. 5B shows the remaining precipitated beads 300 and residual oil before washing, and the circled portion shows the appearance of the residual oil. In addition, the bottom image of FIG. 5B shows the appearance of the beads washed according to this embodiment.

Thereafter, the washed beads are dried at room temperature (S170). FIG. 5C shows the appearance of dried chitosan particles loaded with a freezing-resistant disinfectant, obtained by drying the washed beads at room temperature.

As described above, the method for manufacturing an eco-friendly capsule according to this embodiment is a method for manufacturing an eco-friendly capsule in which particles are manufactured using a biodegradable polymer in order to maintain the effect of a freezing-resistant disinfectant for a long period of time. To this end, in the present invention, in order to manufacture biodegradable particles in a uniform size, a method for manufacturing and spontaneously precipitating and separating an emulsion of microparticles using a microfluidic chip is used.

In addition, unlike the conventional technology in which the process of removing oil remaining in the emulsion is complicated and the loss of beads occurs during the process, the method for manufacturing an eco-friendly capsule according to this embodiment allows the residual oil to be removed simply and effectively by simultaneously using nonionic surfactants of water phase and oil phase types to remove the residual oil.

In addition, in the method for manufacturing an eco-friendly capsule according to this embodiment, when the drying of the washed beads is completed, the dried beads, that is, particles loaded with a freeze-resistant disinfectant, may be manufactured in the form of a powder that may be rehydrated, so that long-term stability or preservation of disinfectant performance may be ensured.

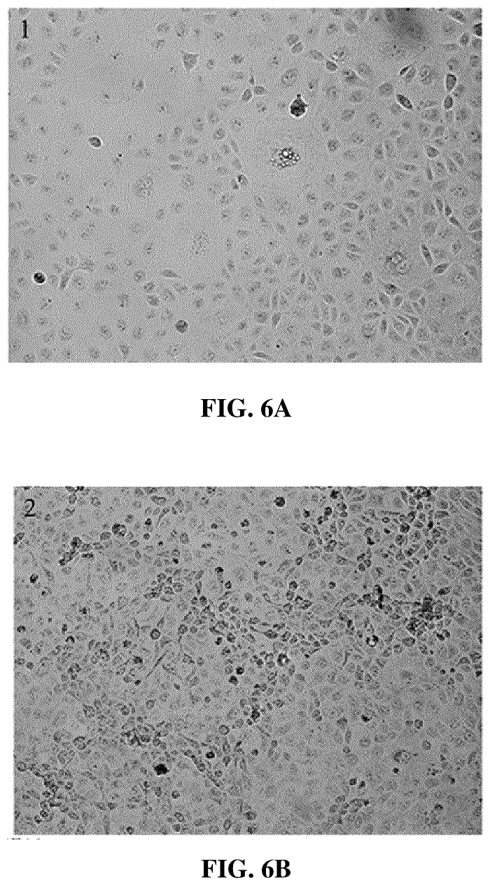

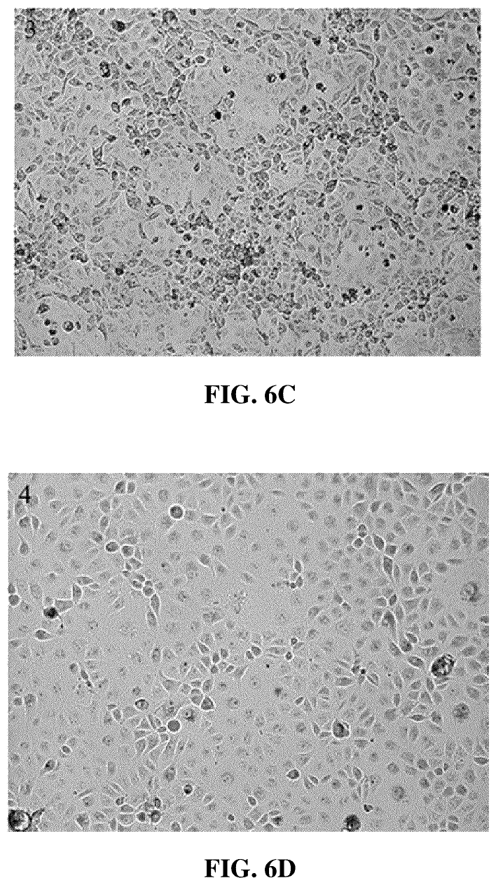

On the one hand, FIGS. 6A to 6D are views illustrating the efficacy of beads produced by varying the content of the cross-linking agent in the method for manufacturing an eco-friendly capsule according to the present invention.

Examination of the efficacy of chitosan particles containing a freezing-resistant disinfectant according to this embodiment was conducted under the condition of containing 20 wt % of chitosan particles and 1 to 15% v/v of GLU as the cross-linking agent 210 in purified water, and FIG. 6A shows a state (X400) in which the cultured cells (MARC-145cell) were observed in a normal form because the disinfectant killed all porcine reproductive and respiratory syndrome viruses in the 1:1 disinfectant (chitosan+disinfectant+beads)-treated sample and the cells were not killed by the porcine reproductive and respiratory syndrome viruses.

FIG. 6B shows a state (X400) in which the cultured cells (MARC-145cell) used were observed in a dead form because the disinfectant did not kill porcine reproductive and respiratory syndrome viruses in the 1:100 disinfectant (chitosan+disinfectant+beads)-treated sample and the cells were killed by the porcine reproductive and respiratory syndrome viruses. In addition, FIG. 6C is a positive control group and shows a state (X400) in which the cultured cells (MARC-145cell) were observed in a dead form because the cells were killed by the porcine reproductive and respiratory syndrome viruses, and FIG. 6D is a negative control group and shows a state (X400) in which the normally cultured cells (MARC-145cell) were observed.

As shown in FIGS. 6A to 6D, as a result, it can be confirmed that a virus reduction rate of 100% (virus reduction rate=[(initial virus content−virus content after reaction)/initial virus content]×100) was shown at a dilution factor of 1:1 of chitosan beads, and the effect was not shown at a dilution factor of more than 1:1 (1:50, 1:100).

In the case of chitosan beads mixed with 15% v/v of GLU as the cross-linking agent 210, there may be a possibility of showing cytotoxicity although virucidal performance is exhibited. Therefore, it was confirmed that the freezing-resistant disinfectant-containing chitosan particles composed of 1 wt % of chitosan, freezing-resistant disinfectant solution and 1% v/v of cross-linking agent by the method for manufacturing an eco-friendly capsule according to this embodiment, had a virucidal effect at a dilution factor of 1:1 to 1:50, as shown in Table 1.

TABLE 1

| | 1:1 | 1:10 | 1:100 | 1:1000 | 1:10000 |
|---|---|---|---|---|---|
| 1:1 | 0/6 | 0/6 | 0/6 | 0/6 | 0/6 |
| 1:50 | 2/4 | 0/6 | 0/6 | 0/6 | 0/6 |
| 1:100 | 6/0 | 4/2 | 3/3 | 0/6 | 0/6 |

| Dilution | | Virus | Disinfectant |
|---|---|---|---|
| Factor | TCID₅₀/mL | Reduction Rate | Effect |
| 1:1 | 0 | 100% | Effective |
| 1:50 | — | — | Effective |
| 1:100 | 2.32 × 10² | 99.89% | Effective |

Although various embodiments of the present invention have been shown and described above, the present invention is not limited to the specific embodiments described above, and it is natural that various modified implementations can be made by those skilled in the art to which the present invention belongs without departing from the gist of the present invention claimed in the claims, and these modified implementations should not be individually understood from the technical spirit or perspective of the present invention.

[Description of Symbols]

| | |
|---|---|
| 11: Water phase material | 100: Emulsion |
| 110: Droplets | 120: Oil phase material |
| 121: Mineral oil | 123: First surfactant |
| 130: Chitosan beads | 200: Water phase solution |
| 210: Cross-linking agent | 220: Second surfactant |
| 300: Precipitated beads | |

1. A method for manufacturing an eco-friendly capsule, the method comprising:
on a microfluidic chip, disposing a water phase material containing chitosan at a first site, and disposing an oil phase material at a second site and a third site;
moving the water phase material and the oil phase material to a reaction site according to a predetermined flow ratio to produce an emulsion containing droplets having a predetermined diameter;
mixing the emulsion with a water phase solution in a mixing tank; and
producing chitosan beads by a reaction of the emulsion with the water phase solution.

2. The method for manufacturing the eco-friendly capsule according to claim 1,
wherein the oil phase material is a material composed by mixing oil and a first surfactant according to a first predetermined ratio, and
the water phase solution is a material composed by mixing a cross-linking agent and a second surfactant according to a second predetermined ratio.

3. The method for manufacturing the eco-friendly capsule according to claim 2, the producing chitosan beads further comprises:
generating oil emulsification at an interface between the emulsion and the water phase solution;
phase-inverting the first surfactant remaining on a surface of the droplets into the second surfactant; and
producing beads by a reaction of the droplets with the cross-linking agent.

4. The method for manufacturing the eco-friendly capsule according to claim 3, during the producing the beads, the reaction of the droplets with the cross-linking agent is promoted by the phase-inverting.

5. The method for manufacturing the eco-friendly capsule according to claim 3, during the producing the beads, the reaction of the droplets with the cross-linking agent is promoted by centrifugation.

6. The method for manufacturing the eco-friendly capsule according to claim 3, further comprising:
moving oil remaining in the water phase solution to the interface by the second surfactant; and
curing and precipitating the produced beads according to a continuous progress of cross-linking.

7. The method for manufacturing the eco-friendly capsule according to claim 6, further comprising:
separating and washing the precipitated beads from the mixing tank; and
drying the washed beads at room temperature.

8. The method for manufacturing the eco-friendly capsule according to claim 1, wherein the predetermined flow ratio between the water phase material and the oil phase material is in a range of 1 to 5-20, and a diameter of the droplets is size-adjustable within 50 μm.

9. An eco-friendly capsule by being manufactured by the method according to claim 1.
